(19) United States
(12) Patent Application Publication
(10) Pub. No.: US 2010/0191155 A1
(43) Pub. Date: Jul. 29, 2010

(54) Apparatus for Calculating Calories Balance by Classifying User's Activity

(75) Inventors: Ig-Jae KIM, Seoul (KR); Hyoung Gon KIM, Seoul (KR); Sang Chul AHN, Seoul (KR)

Correspondence Address: FENWICK & WEST LLP, SILICON VALLEY CENTER, 801 CALIFORNIA STREET, MOUNTAIN VIEW, CA, 94041, US

(73) Assignee: Korea Institute of Science and Technology, Seoul (KR)

(21) Appl. No.: 12/535,630

(22) Filed: Aug. 4, 2009

(30) Foreign Application Priority Data
Jan. 28, 2009 (KR) 10-2009-0006635

(51) Int. Cl. A61B 5/11; G06K 7/00; G06K 7/10

(52) U.S. Cl. 600/595; 235/439; 235/462.41

(57) ABSTRACT

An apparatus for calculating calorie balance based on an activity classification, disclosed herein, includes a calculation part calculating characteristic values of acceleration and a user's calorie expenditure from the user's activities, and calculating food data and the user's calorie intake from foods taken by the user; and a recognition part recognizing the user's activities based on the characteristic values of acceleration, and recognizing the foods based on the food data. The characteristic values of acceleration are extracted from acceleration data of acceleration sensors, which determine the user's activities, and include information on the relationship between the acceleration data and the user's activities. The calculation part calculates calorie balance, using the user's calorie expenditure and the user's calorie intake.

### CROSS-REFERENCE TO RELATED APPLICATION

This application claims priority to Republic of Korea Patent Application No. 10-2009-0006635, filed on Jan. 28, 2009, and all the benefits accruing therefrom under 35 U.S.C. §119(a), the contents of which in its entirety are herein incorporated by reference.

### BACKGROUND

1. Field

This disclosure relates to an apparatus for calculating calorie balance based on classified information on a user's activity, which may be applied in a mobile environment. More specifically, the apparatus is disclosed herein for calculating calorie balance by measuring the user's calorie expenditure from the user's activity recognized from acceleration data obtained by acceleration sensors, and by measuring the user's calorie intake from a food recognized from an image of the food taken by a camera.

2. Description of the Related Art

Healthcare requires the measurement of intake and expenditure of calories. The calorie expenditure is measured by determining basal metabolism of an individual, thermic effect of exercise, and thermic effect of food.

The basal metabolism refers to the minimum amount of energy needed to survive. That is, the basal metabolism corresponds to the amount of energy expended for a process of metabolism for a basal life activity such as maintaining body temperature, breathing, and heart beating. Generally, the amount of energy as much as the basal metabolism is expended, when resting or not moving. The basal metabolism may be calculated automatically from such variables as body weight and age.

The thermic effect of exercise refers to the amount of energy expended through various activities including walking and running in a day except when an individual sleeps or rests.

And, the thermic effect of food refers to the amount of energy required for processing of foods taken, such as digestion, absorption, and transfer. The thermic effect of food is known to account for about 10% of the sum of basal metabolism and thermic effect of exercise.

Together with measuring the calorie expenditure, it is important to calculate the actual amount of calorie taken by a user. Any method for automatically determining a kind of food taken by the user has not been embodied yet. As a method for recognizing what food the user has taken, it has been used to directly input the food taken.

Recently, mobile technology has been developed, and a mobile phone is equipped with several digital sensors including acceleration sensor, GPS, and camera, so that the sole phone makes it possible to trace the user's activity and location and get related images.

An apparatus is in request for informing a state of calorie balance by easily calculating the user's calorie intake and expenditure in a mobile environment.

### SUMMARY

There is provided an apparatus for monitoring the state of metabolism of a user by means of automatically calculating the calorie intake and expenditure using a mobile device such as a mobile phone.

The apparatus for calculating calorie balance based on an activity classification, according to the embodiment, comprises a calculation part calculating characteristic values of acceleration and a user's calorie expenditure from the user's activities, and calculating food data and the user's calorie intake from foods taken by the user; and a recognition part recognizing the user's activities based on the characteristic values of acceleration, and recognizing the foods based on the food data. The characteristic values of acceleration are extracted from acceleration data of acceleration sensors, which determine the user's activities, and include information on the relationship between the acceleration data and the user's activities. The calculation part calculates calorie balance, based on the user's calorie expenditure and the user's calorie intake.

Because a mobile device is always carried along by a user, continuous detection of the user's activities is possible to analyze information relating to calories, so as to determine accurately the expenditure of calories. Moreover, the method according to the embodiment for calculating the calorie expenditure based on kinds of activities is improved in accuracy, compared with conventional methods based on the calorie expenditure manually inputted or the analysis of a pattern of walking.

Because a history of foods taken is recorded automatically by taking photographs of the foods such as beverages and snacks using the mobile device, it becomes easy to calculate the calorie intake and provide a new environment to people who want to regulate their eating habits.

### BRIEF DESCRIPTION OF THE DRAWINGS

The above and other aspects, features and advantages of the disclosed exemplary embodiments will be more apparent from the following detailed description taken in conjunction with the accompanying drawings.

FIG. 1 is a flow chart explaining the operation of an apparatus for calculating calorie balance according to an embodiment.

FIG. 2 is a flow chart explaining the process for calculating the calorie expenditure classified by each of activities, which is part of the flow chart of FIG. 1.

FIGS. 3A, 3B, 3C, and 3D represent the measurements of the calorie expenditure classified by each of activities.

FIG. 4 represents the calorie expenditure in the state of standing among several activities.

FIG. 5 is a flow chart explaining the process for calculating the calorie intake classified by each of foods taken, which is part of the flow chart of FIG. 1.

FIG. 6 illustrates the apparatus for calculating calorie balance according to the embodiment.

FIG. 7 explains the process for extracting characteristic values of acceleration according to an embodiment.

FIGS. 8A and 8B illustrate an embodiment of the apparatus for calculating balance according to the invention embodied on a computer.

### DETAILED DESCRIPTION

Exemplary embodiments now will be described more fully hereinafter with reference to the accompanying drawings, in which exemplary embodiments are shown. This disclosure may, however, be embodied in many different forms and should not be construed as limited to the exemplary embodiments set forth therein. Rather, these exemplary embodiments are provided so that this disclosure will be thorough and complete, and will fully convey the scope of this disclosure to those skilled in the art. In the description, details of well-known features and techniques may be omitted to avoid unnecessarily obscuring the presented embodiments.

The terminology used herein is for the purpose of describing particular embodiments only and is not intended to be limiting of this disclosure. As used herein, the singular forms “a”, “an” and “the” are intended to include the plural forms as well, unless the context clearly indicates otherwise. Furthermore, the use of the terms a, an, etc. does not denote a limitation of quantity, but rather denotes the presence of at least one of the referenced item. The use of the terms “first”, “second”, and the like does not imply any particular order, but they are included to identify individual elements. Moreover, the use of the terms first, second, etc. does not denote any order or importance, but rather the terms first, second, etc. are used to distinguish one element from another. It will be further understood that the terms “comprises” and/or “comprising”, or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

In the drawings, like reference numerals in the drawings denote like elements. The shape, size and regions, and the like, of the drawing may be exaggerated for clarity.

Explained hereafter is an apparatus for calculating calorie balance with reference to the accompanying drawings.

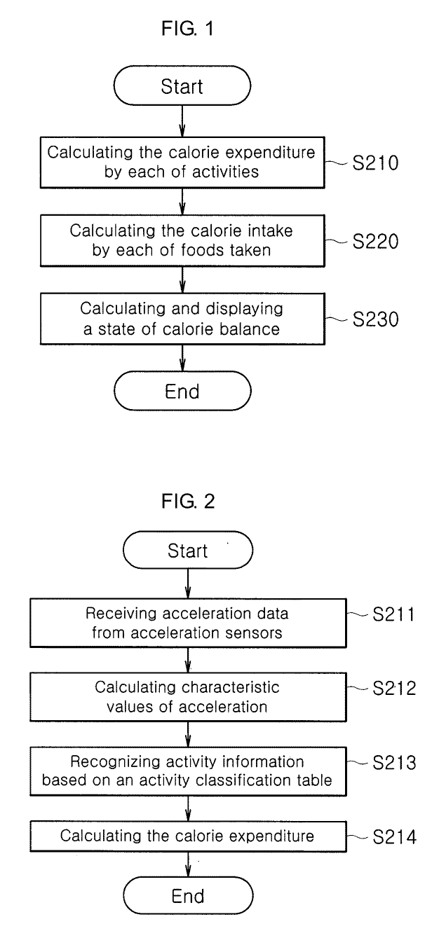

FIG. 1 is a flow chart explaining an operation of the apparatus 100 for calculating calorie balance according to an embodiment. The expenditure of calories classified by a user's activity is calculated (S210), the intake of calories classified by a food taken is calculated (S220), and the state of calorie balance, which is calculated using the expenditure and the intake of calories each calculated at the steps S210 and S220, may be displayed (S230). The apparatus for calculating calorie balance may calculate the difference between the calorie expenditure and the calorie intake, and then inform to the user the state of calorie balance in order for him or her to care for his or her health by adjusting diet and exercise.

FIG. 2 is a flow chart explaining the process of calculating the expenditure of calories classified by the user's activity. The step S210, which calculates the expenditure of calories by the activity, includes receiving (S211) acceleration data from acceleration sensors, and calculating (S212) a characteristic value of acceleration from the acceleration data. The characteristic value of acceleration is extracted from the acceleration data, and is an essential factor to recognize information on the user's activity. That is, the characteristic value of acceleration is a clue to recognize the user's activity. The characteristic value may be average acceleration, a value of energy, a correlation, or a value of entropy, etc.

From an activity classification table established with a database, the user's activity information is recognized based on the characteristic value of acceleration (S213). “The activity information” means herein the user's activity and the amount of calories consumed by that activity. The activity classification table is a database established by repeated learning, which includes several kinds of activities classified by the characteristic value of acceleration. A food classification table stores information on the calorie expenditure by several kinds of activities.

For example, while a subject equipped with the acceleration sensors performs such activities as walking, running, and/or sitting, etc, acceleration data is measured from the activity, and a characteristic value of acceleration is extracted from the acceleration data. In this way, the activity classification table may be achieved by matching activity and its characteristic value of acceleration. The more kinds of the characteristic values of acceleration are, the more specific the activity classification table becomes and the more precise the recognition of the activity becomes. The calorie expenditure by various activities may be calculated at the same time while the activity classification table is established.

After the user's activity is recognized, the calorie expenditure by activities may be searched from the activity classification table. The recognition part searches the calorie expenditure corresponding to the user's activity recognized from the activity classification table, and calculates the calorie expenditure corresponding to the user's activity (S214).

Explained hereafter is an embodiment of a method for establishing the activity classification table. It was explained above that the activity classification table is prepared to store the activity information and may be established through one process. The activity information includes an activity classified by the characteristic value of acceleration, and the calorie expenditure by that activity.

A method known to the most precisely measure the calorie expenditure is to use the ratio of oxygen intake to carbon dioxide outflow. For this method, a gas exchange system may be used. After having the user equipped with acceleration sensors perform several kinds of activities, the calorie expenditure by an activity may be measured accurately by obtaining a correlation between the acceleration data from the acceleration sensors and the calorie expenditure measured by the gas exchange system. That is, only with the acceleration sensors provided to the user, a relatively precise measurement of the calorie expenditure may be accomplished. Recently, mobile phones have been already provided with those acceleration sensors, so the user's activity may be recognized at any time by a handy mobile phone to calculate the calorie expenditure.

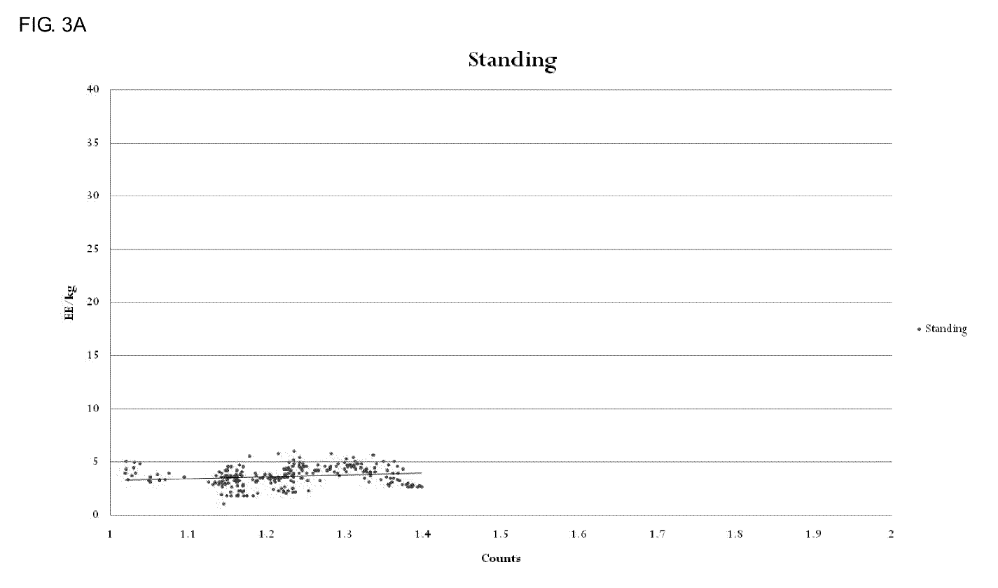

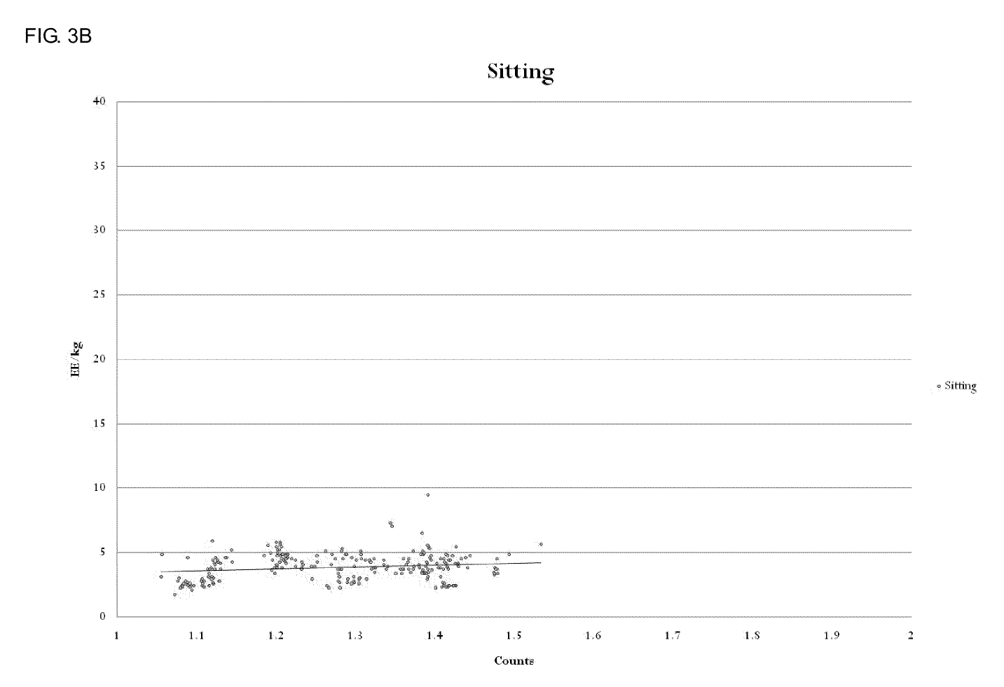

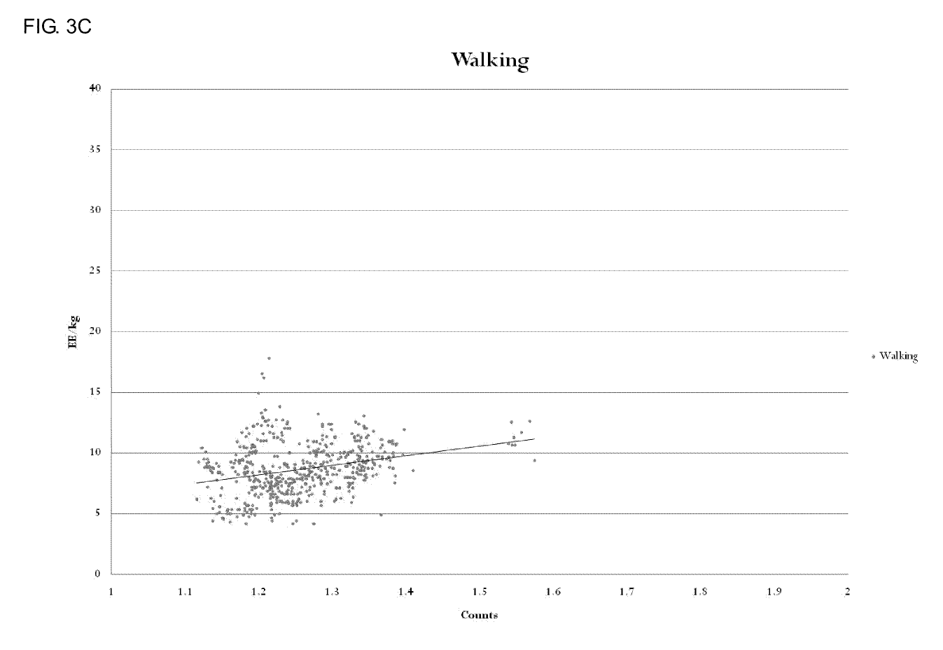

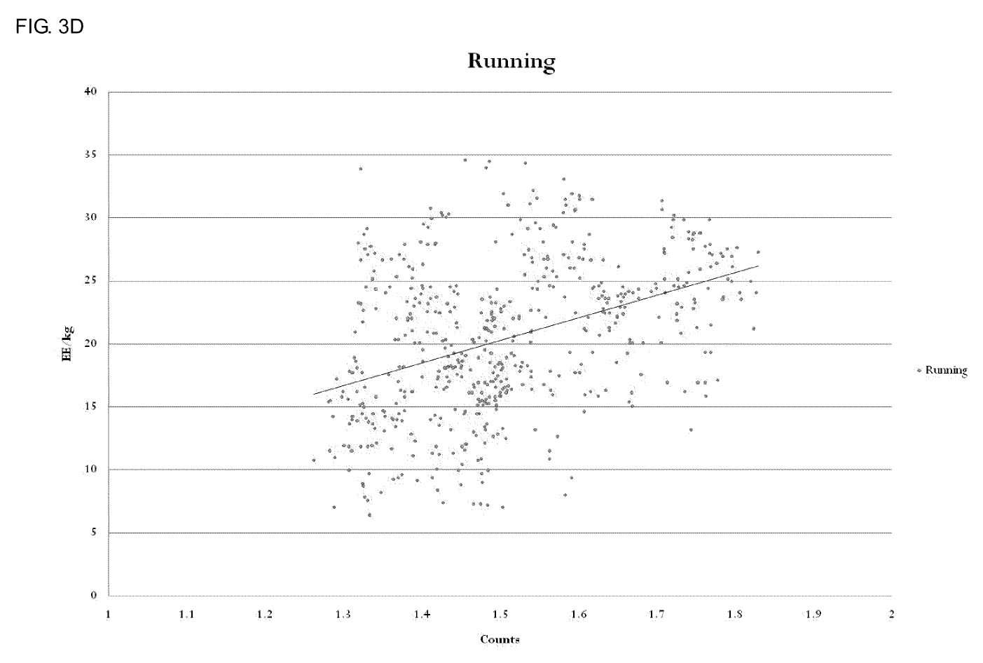

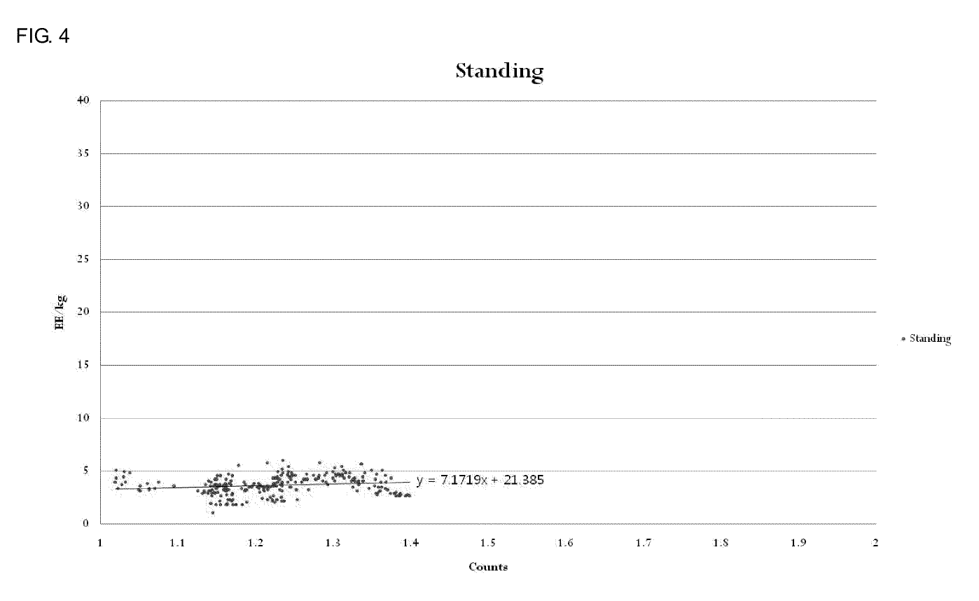

FIGS. 3A, 3B, 3C, and 3D illustrate the relationship between the data of acceleration sensors and the calorie expenditure measured by the gas exchange system. The figures show the distributions of the calorie expenditure for various activities (standing, sitting, walking, running). FIG. 4 depicts the distribution based on the data of the calorie expenditure measured when 17 subjects equipped with the gas exchange system and the acceleration sensors perform several kinds of activities.

In the figure, the count is

$${\int }_{10 sec} ( \vert {a}_{x} \vert + \vert {a}_{y} \vert + \vert {a}_{z} \vert ) d t$$

i.e., an integral of the absolute values of acceleration values of a triaxial acceleration sensor over 10 seconds. The period of time 10 seconds may be alternated. The factor EE/kg means the energy expenditure per 1 kg, and has the unit of calories.

FIG. 4 exemplifies an algebraic expression of the calorie expenditure according to the acceleration value by an activity, which is obtained from the value of calories precisely calculated from the gas exchange system. The calorie expenditure (EE/kg) according to the count corresponding to the acceleration value approximates to a linear equation. A higher order fitting algorithm may be used to draw a more precise equation.

Using the resultant equation, the calorie expenditure of the user may be precisely measured from the acceleration value stored in a mobile phone only by inputting the user's body weight to the phone. General-purpose acceleration sensors may be used to calculate the calorie expenditure from the corresponding equation, so the embodiment herein is not limited to the mobile phone in which the acceleration sensors are mounted.

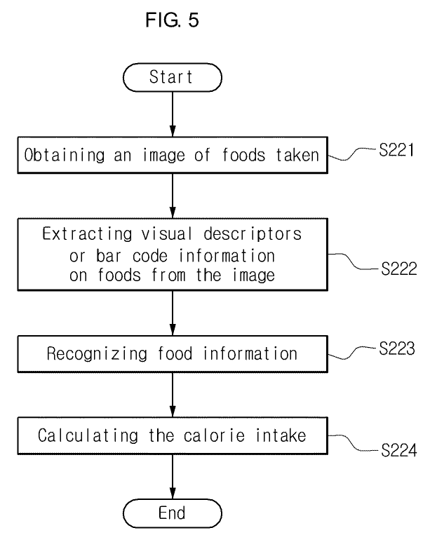

FIG. 5 is a flow chart explaining the process for calculating (S220) the calorie intake classified by foods of FIG. 1. The image of foods taken by a user is obtained from a camera (S221), and visual descriptors or information on a bar code of the foods is extracted from the obtained image (S222). The image information of foods taken by the user is obtained using the camera, and, at the same time, the information on foods taken may be recognized (S223) by an image processing of the above obtained image. Further, the food information may be recognized from RFID tag information, using an RFID reader. The food information includes a kind of food and the amount of calories of the food.

The visual descriptor characterizes an object recorded in such an image as a photograph, and provides a characteristic visual display which is distinguished from other objects. It is similar to the concept of keyword in text-based search. Using the scale invariant feature transform (SIFT) algorithm, the visual descriptor may be extracted from the obtained image. The visual descriptor makes it possible to recognize an object in a fast and precise way.

When reorganization of foods by the bar code information, the visual descriptors, or the RFID tag information is finished, the amount of calories of food intake is searched in the activity classification table. Several kinds of foods are widely known in terms of their calories, and the data on the calories may be stored in a storage. The calorie intake by the user may be calculated (S224) using the food intake and the amount of calories of that food.

Recent mobile phones basically have a camera mounted therein, and those having an RFID reader built in are also being marketed. So, the services explained above may be embodied in one mobile device., e.g. a mobile phone. This makes it possible to realize an apparatus for calculating calorie balance only with a mobile phone, which is always carried along, without any additional device.

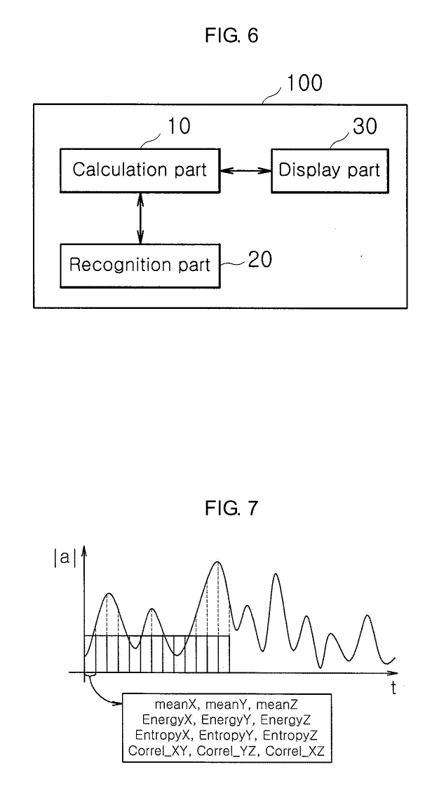

FIG. 6 is a diagram explaining an apparatus 100 for calculating calorie balance according to an embodiment. The apparatus 100 may include a calculation part 10 calculating a characteristic value of acceleration and the calorie expenditure by a user from the user's activity, and calculating food data and the calorie intake by the user from the food taken by the user; and a recognition part 20 recognizing the user's activity based on the characteristic value of acceleration, and recognizing the food based on the food data. The characteristic value of acceleration is obtained from acceleration data of acceleration sensors, which measure the user's activities. The value includes information on a relationship between the acceleration data and the user's activity.

The calculation part 10 may calculate the characteristic value of acceleration, using the acceleration data received from the acceleration sensors. The acceleration sensors are triaxial acceleration sensors. The characteristic value means an average acceleration, an energy value, a correlation and an entropy value.

The process for obtaining each of the characteristic values is as follows. FIG. 7 shows a motion unit. As illustrated in FIG. 7, sample windows are generated with 256 pieces of data, which may be variably adjusted. Each of the windows consists of four segments, one of which corresponds to one second. During one second, 64 samples may be extracted. The number of samples per second may be changed according to a kind of the system. Based on the samples for four seconds, i.e. 256 samples with the sampling of 64 Hz, characteristic values, e.g. average accelerations with respect to x-, y-, and z-axes (meanX, meanY, meanZ), energy values (EnergyX, EnergyY, EnergyZ), entropy values (EntropyX, EntropyY, EntropyZ), and correlations (Correl_XY, Correl_YZ, Correl_XZ), may be obtained. Further, when continuous sample windows overlap by 128 samples and move, the samplings may be overlapped over the range of the sample windows overlapped. In this way, the discreetness of sampling may be reduced and continuity may be maintained.

Specifically, in order to obtain the characteristic values, a fast Fourier transform (FFT) is applied to the absolute value of the acceleration data. The average accelerations among the characteristic values may be easily obtained by extracting the DC element of the sample windows. This means an average acceleration value over an interval of sample windows.

After applying FFT, all values except the DC element are, respectively, squared, and then summed. The outcome is divided by the size of the window, thereby resulting in a standardized value as a value of energy.

The correlations mean correlations between the acceleration values of each axis. The correlation of x-y axes, y-z axes, or z-x axes may be calculated as follows.

Correl_{—}XY=acc_{—}x*acc_{—}y

Correl_{—}YZ=acc_{—}y*acc_{—}z

Correl_{—}XZ=acc_{—}x*acc_{—}z

The equation is repeatedly calculated so as to meet the sampling rate, and then the summed amount is divided by the number of samples. This is the characteristic value for understanding a relationship between x, y and z axes of the acceleration sensors. The entropy values are obtained by calculating the entropy information in which all of the values except the DC element are standardized. Continuous sample windows overlap and move by the unit of 128 samples (in case of sample window of 256 pieces of data), and each of sample windows means an interval of 4 seconds.

The entropy value is calculated by the following equation.

$$Info Entropy = - {\sum }_{i = 1}^{n} p ( {x}_{i} ) {log}_{2} p ( {x}_{i} )$$

The factor p(xᵢ) means the rate which is obtained in the way in which the number corresponding to a bin, with respect to all of the values except the DC element after the absolute value of acceleration is computed through a FFT algorithm, is divided by the number of the absolute value of the whole accelerations or acceleration data. The bin means a value to which the absolute value of acceleration approximates. For example, if the absolute values of accelerations are within the range of 0 to 10, each of the absolute values may be allotted to one of ten bins, i.e., {0, 1, 2, 3, 4, 5, 6, 7, 8, 9}. Here, the factor p(x₁) is the probability at which acceleration data corresponding to the range between 0 and 1 of the absolute value of the acceleration is generated. That is, the expression may be p(x₁)=[0,1]_{|a|}/(the number of the whole acceleration data). Here, the term [0,1]_{|a|} is the number of cases in which the acceleration data having the absolute value of the range between 0 and 1 is generated. As such, the entropy values may be calculated using the distribution probability of the acceleration data. With the entropy values calculated as above, the relationship between the activities and the entropy values may be obtained, thereby making it possible to recognize the user's activity.

Referring back to FIG. 6, the calculation part 10 may calculate the user's calorie expenditure, using the information on the user's activity recognized as above. Further, the calculation part 10 may calculate the user's calorie intake, using the food information recognized by the recognition part 20, which will be explained below in detail. Moreover, the calculation part 10 may calculate calorie balance, using the user's calorie intake and calorie expenditure.

The recognition part 20 may recognize the information on the user's activities based on the characteristic values of acceleration. The recognition part 20 proceeds to recognize the activity with reference to the activity classification table based on these characteristics. In this way, the user's activities, such as walking, running, lying, and standing, may be recognized with more than 90% accuracy, when he or she wears the acceleration sensors on his or her waist. If the user wears the sensors on his or her wrist, the movements of the hand may be classified.

Further, the recognition part 20 may recognize the foods taken by the user, using the food data extracted from the RFID or the images of the foods. The food data is obtained from the images of the foods, and contains the visual descriptors or barcode information on the foods. The food data may also be extracted from the RFID tags, and contains the tag information on the foods.

The recognition part 20 may recognize the foods, using the food classification table. The food classification table classifies several foods according to the food data, and stores the amount of calories of several foods. In short, the recognition part 20 obtains the food data of the foods taken by the user, using the RFID tags or the images of the foods taken by the users, and finds the foods corresponding to the food data from the food classification table, and then recognizing the food being taken by the user.

Meanwhile, the apparatus may further include a display part 30, which displays excessive calories if the user's calorie intake is more than the calorie expenditure, insufficient calories if the intake is less than the expenditure, and balanced calories if the intake is the same as the expenditure.

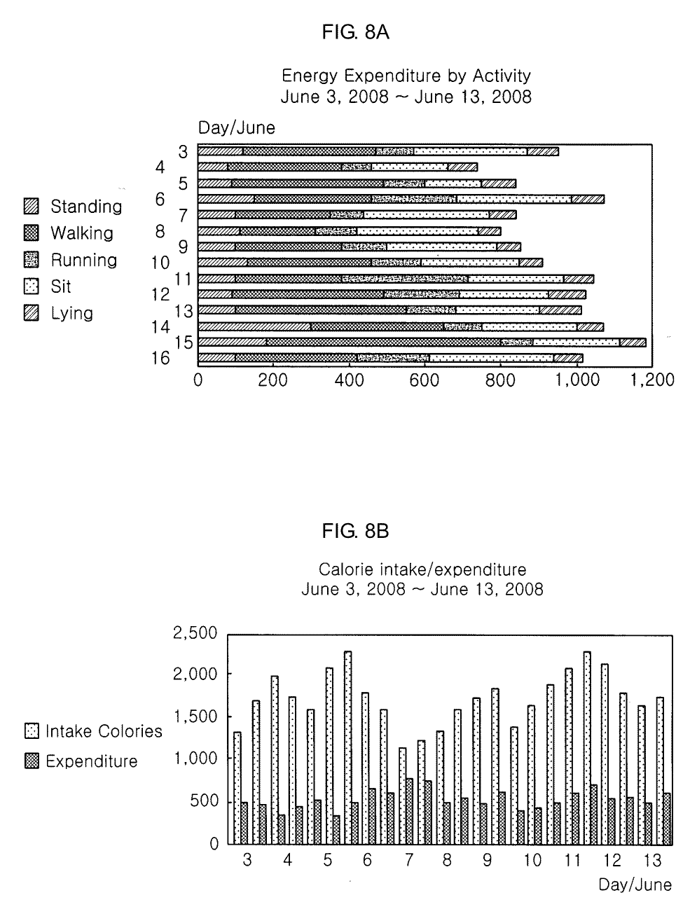

FIGS. 8A and 8B show an embodiment of the apparatus for calculating calorie balance, which, using a GPS built in or equipped outside a mobile phone, allows a user, at any time he or she wants, to search for the information on when, where and what he or she ate, and how much calories he or she consumed. The apparatus for calculating calorie balance according to the embodiment can represent an image in which routes through which the user has moved during a particular period of time in a day are displayed, and in which the activities of the user including running, standing, walking, and sitting are recognized and displayed. FIG. 8A is an example of a graph illustrating by the day the calorie expenditure of the recognized activities (running, standing, walking, sitting, etc). FIG. 8B is an example of a graph representing the calorie intake compared with the calorie expenditure by the day to help understanding the user's health or nutritional state.

Moreover, when a target calorie intake that the user wants to achieve is inputted, the mobile phone may act as a virtual health manager for the user.

While the exemplary embodiments have been shown and described, it will be understood by those skilled in the art that various changes in form and details may be made thereto without departing from the spirit and scope of the present invention as defined by the appended claims.

In addition, many modifications can be made to adapt a particular situation or material to the teachings of the invention without departing from the essential scope thereof Therefore, it is intended that the present invention not be limited to the particular exemplary embodiments disclosed as the best mode contemplated for carrying out this invention, but that the present invention will include all embodiments falling within the scope of the appended claims.

What is claimed is:

1. An apparatus for calculating calorie balance based on an activity classification, comprising:
a calculation part calculating a characteristic value of acceleration and a user's calorie expenditure from the user's activity, and calculating food data and the user's calorie intake from a food taken by the user; and
a recognition part recognizing the user's activity based on the characteristic value of acceleration, and recognizing the food based on the food data,
wherein the characteristic value of acceleration is extracted from acceleration data of acceleration sensors, which determine the user's activity, and include information on the relationship between the acceleration data and the user's activity, and
wherein the calculation part calculates calorie balance, using the user's calorie expenditure and the user's calorie intake.

2. The apparatus according to claim 1, wherein the food data is extracted from an image of the food taken by a camera, and is a visual descriptor or bar code information of the food.

3. The apparatus according to claim 1, wherein the food data is extracted from an RFID tag of the food obtained by an RFID reader, and is tag information of the food.

4. The apparatus according to claim 1, wherein the characteristic value of acceleration comprises an average acceleration,
wherein the average acceleration is obtained by extracting a DC element through a fast Fourier transform of the acceleration data.

5. The apparatus according to claim 1, wherein the characteristic value of acceleration comprises an energy value,
wherein the energy value is obtained by a process in which all of the values, except a DC element, which are calculated through a fast Fourier transform of the acceleration data, are respectively squared and summed, and then divided by the number of the acceleration data.

6. The apparatus according to claim 1, wherein the characteristic value of acceleration comprises a correlation,
wherein the correlation is a correlation between acceleration data for each of axes of the acceleration data.

7. The apparatus according to claim 1, wherein the characteristic value of acceleration comprises an entropy value,
wherein the entropy value is obtained using a distribution probability of the absolute value of the acceleration data.

8. The apparatus according to claim 1, wherein the recognition part recognizes the user's activity, referring to an activity classification table,
wherein the table is prepared to have several activities classified by the characteristic value of acceleration, and to store information on the amounts of calories consumed by the several activities.

9. The apparatus according to claim 8, wherein the activity classification table stores the results that measure several activities and characteristic values of acceleration corresponding to the several activities by repeated learning.

10. The apparatus according to claim 9, wherein the calculation part calculates the user's calorie expenditure, using the recognized user's activity and the amount of calorie stored in the activity classification table.

11. The apparatus according to claim 1, wherein the recognition part recognizes the foods, using a food classification table,
wherein the food classification table is prepared to have several foods classified by the food data, and to store information on the amounts of calories of the several foods.

12. The apparatus according to claim 11, wherein the calculation part calculates the user's calorie intake, using the recognized food and the amount of calories of the food.

13. The apparatus according to claim 10, further comprising a display part displaying the state of calorie balance based on the difference between the user's calorie intake and the user's calorie expenditure.

14. The apparatus according to claim 13, wherein the display part displays excessive calories if the user's calorie intake is more than the user's calorie expenditure, insufficient calories if the user's intake is less than the user's expenditure, and balanced calories if the user's intake is the same as the user's expenditure.
